(19) United States
(12) Patent Application Publication
(10) Pub. No.: US 2006/0134190 A1
(43) Pub. Date: Jun. 22, 2006

(54) Formulations of bisphosphonate drugs with improved bioavailability

(75) Inventors: Tae Kyoung Kim, Winston-Salem, NC (US); Aqeel A. Fatmi, Greensboro, NC (US)

Correspondence Address: Charles T. Joyner, 7515 Johnson Mill Rd., Bahama, NC, 27503, US

(73) Assignee: Banner Pharmacaps Inc.

(21) Appl. No.: 11/014,252

(22) Filed: Dec. 16, 2004

(51) Int. Cl. A61K 31/663; A61K 9/48; A61K 9/20

(52) U.S. Cl. 424/451; 424/464; 514/102

(57) ABSTRACT

This invention relates to formulations of bisphosphonates such as alendronate. The formulations taught herein enhance bioavailability of bisphosphonates and reduce esophageal and gastric ulcerations associated with them. Also taught herein are methods of preparing the formulations and their clinical use in the treatment of osteoporosis and other bone diseases.

### BACKGROUND OF THE INVENTION

Bone resorption is the destructive erosion of the bone matrix by loss of minerals and fragmentation of the collagen. Upon activation, osteoclasts increase bone resorption by secreting acids and digestive protease. A decline or withdrawal of androgen and estrogen leads to excessive osteoclastic activity resulting in accelerated bone resorption. Osteoblasts are bone forming cells derived from precursor cells in the blood. The Osteoblasts migrate to areas of resorption and deposit collagen and minerals to remodel the eroded area. Bones undergo constant erosion by osteoclastic activity intimately coupled with remodeling by osteoblastic activity.

Osteoporosis is the loss of bone density through a destructive physiological cascade that exceeds the normal osteoblastic remodeling activity. Tumor cells have an important role in osteoporosis. The tumor cells within the bone also produce transforming growth factor (TGF)-β which stimulates release of parathormone (PTH) and matrix metalloprotease-2 (MMP-2), which dissolves collagen 1. MMP-2 can indirectly stimulate osteoblast growth.

Strategies for prevention and reversal of osteoporosis and related diseases include treatment with anabolic steroids, selective estrogen receptor modulators, mineral supplements, and thyroid and parathyroid hormones. In particular, potent, direct inhibitors of osteoclast-mediated bone resorption, such as bisphosphonates, i.e., derivatives of bisphosphonic acids, and analogues of pyrophosphate, have been successfully used in treating osteoporosis, as well as Paget's disease of bone, hypercalcemia and osteolytic bone disease or malignancy, primary and secondary hyperparathyroidism, and breast cancer.

Several bisphosphonates and analogs of pyrophosphate are available or in clinical trials as anti-resorption drugs. Alendronate (chemically named, 4-amino-1-hydroxybutyliden-1,1-bisphosphonic acid trihydrate monosodium), e.g. Fosamax, trademark of Merck, is the most frequently prescribed bisphosphonate. Unfortunately, bisphosphonates administered orally are rapidly degraded by gastric fluids substantially reducing the amount that enters the intestines and is absorbed into the body. Thus, extremely low bioavailability is a problem for solid oral dosage formulations of bisphosphonates.

Bisphosphonates can strongly chelate most divalent cations including Ca²⁺ and Mg²⁺ originating from food or inter- and intracellular matrices. Consequently, by chelation they can tear out intercellular Ca²⁺, triggering the ulceration process. Further, absorption of oral dosages of bisphosphonates is significantly inhibited by the presence of food containing calcium or other divalent ions. Therefore, while being treated with bisphosphonate drugs, patients need special diets and must avoid beverages that contain divalent cations, such as mineral water and orange juice. Typically, patients are advised not lie down for 30 minutes after administration of a bisphosphonate drug, such as alendronate, because of the risk of ulceration of the esophageal tract.

Although bisphosphonate drugs are useful for the prevention and treatment of osteoporosis and other bone diseases, their bioavailability limits their clinical usefulness. Therefore, there is a need for oral dosage forms of these drugs that substantially boost bioavailability and avoid esophageal and gastric ulcerations.

### SUMMARY OF THE INVENTION

The present invention provides a bioavailable pharmaceutical formulation of a bisphosphonate or pharmaceutically acceptable salt thereof. The first aspect of the invention is a formulation comprising a therapeutic amount of a bisphosphonate or pharmaceutically acceptable salt thereof temporarily complexed with a pharmaceutically acceptable complexing agent, and optionally one or more pharmaceutically acceptable excipients and optionally one or more pharmaceutically acceptable carriers. The formulation may be in the form of a solid, solution, suspension, gel, emulsion, or paste. In one embodiment of this aspect the bisphosphonate is alendronate and the complexing agent comprises at least one trivalent metal ion selected from trivalent manganese, iron and cobalt. In another embodiment, the formulation is encapsulated in a soft gelatin capsule, optionally having enteric properties.

In a second aspect, the invention provides a bioavailable pharmaceutical formulation comprising a therapeutic amount of a bisphosphonate or pharmaceutically salt thereof, one or more oils, one or more waxes, or a combination thereof, a surfactant, and optionally water. The formulation of this aspect is encapsulated in a soft gelatin capsule, optionally having enteric properties.

In a third aspect, the invention provides a method of treatment of a patient suffering from a bone resorption disease comprising administering to the patient an osteogenically effective amount of a formulation of the first or second aspects.

In a fourth aspect, the invention provides a method of manufacturing the formulation of the first and second aspects comprising encapsulation of a solution, gel, emulsion, or paste containing a therapeutic amount of a bisphosphonate or pharmaceutically salt thereof within a soft gelatin capsule optionally having an enteric properties.

### DETAILED DESCRIPTION OF THE INVENTION AND EMBODIMENTS

The formulations of the present invention are useful for the treatment and prevention of bone resorption, i.e. bone loss, such as that associated with osteoporosis. Therefore, a patient in need of the formulations of the present invention would be a patient suffering from a bone loss disease. In addition to osteoporosis, other bone diseases include osteoarthritis, Paget's disease, osteomalacia, multiple myeloma and other forms of cancer, steroid therapy, and age related loss of bone mass. A patient may also suffer bone loss as a result of bone fracture, non-union defect, and similar bone flaws and failures.

The present formulations are intended for oral administration and are applicable for the treatment of mammals, especially human and particularly post-menopausal females. The method of treatment of the present invention prescribes administration of an osteogenically effective amount of a bisphosphonate; in particular trihydrated alendronate monosodium, via a formulation described herein to a patient suffering from, or in danger of suffering from, a bone loss disease. The formulations of the present invention improve the bioavailability and reduce severe side effects of bisphosphonate drugs in general and alendronate in particular.

In certain of the formulations, bisphosphonates weakly chelate trivalent cations which temporarily mask excessive anionic charges on the bisphosphonic acid moieties and partially expose the cationic charges to render the bisphosphonates more accessibility toward anionic epithelial cell membrane. Therefore, the trivalent cations are more efficient delivery systems than the strongly chelated divalent cations such as Ca²⁺. Enteric properties are imparted in the formulations by adding one or more agents to lower the pH below 7 and preferably, in the range of about 5 to about 6.5. The temporarily chelated bisphosphonates may be blended with hydrosoluble carriers to form suspensions or gels for filling soft gelatin capsules, or they can be used to prepare solid formulations such as tablets and solid filled capsules.

The present formulations avoid ulceration of the esophageal tract and stomach lining, by masking the bisphosphonic acid moiety and substantially reducing its tendency to tear out intercellular Ca²⁺. The formulations can also serve as a delivery system for physiologically essential metals as trivalent ions such as, nickel (Ni³⁺), titanium (Ti³⁺), vanadium (V³⁺), manganese (Mn³⁺), iron (Fe³⁺) and cobalt (Co³⁺), and trace metals.

Alternatively, certain other formulations of the present invention improve bioavailability by suspending one or more bisphosphonates, e.g. alendronate, in one or more pharmaceutically acceptable hydrophobic carrier materials such as oils, waxes, or combination of oils and waxes in the presence of a pharmaceutically acceptable surfactant. Optionally, water can be added to the formulation to form a semisolid such as an emulsion or paste. The resulting suspensions and semisolids can be filled into soft gelatin capsules.

Bisphosphonic acid compounds have the common structure of formula (I), 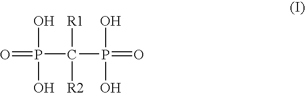
Wherein R¹ is selected from the group consisting of hydrido, hydroxyl, alkoxy and halo, and R² is selected from the group consisting of halo, —(CH₂)ₘ—NR³R⁴, —(CH₂)ₙ—R⁵, —O—R⁶ and —S—R⁷ wherein m is an integer in the range of zero to 8, n is an integer in the range of 1 to 4, R³ and R⁴ are independently hydrogen or alkyl, or together form a C⁵-C⁷ cyclic group, and R⁵, R⁶ and R⁷ are independently aryl or heteroaryl and may be either unsubstituted or substituted with one or more substituents, such as chloro.

As used herein, “bisphosphonate” and “bisphosphonate drug” mean a derivative of bisphosphonic acid known in the art for remodeling bone. Examples include, but are not limited to, alendronate, risedronate, etidronate, clodronate, pamidronate, tiludronate, ibandonate, zoledronate, Incadronate, olpadronate, neridronate, or amidronate. The term “active agent” refers to orally deliverable bisphosphonates. Except where noted otherwise, the term “bisphosphonate” also includes pharmaceutically acceptable salt thereof.

The term “carrier” in the present context means pharmaceutically acceptable material(s) that provides body and flow characteristics to the formulation. Certain formulations of the present invention employ either hydrophobic (lipophilic), or hyrosoluble carriers.

Hydrophobic carriers used herein include animal, vegetable, or mineral oils or waxes, including combinations thereof, so long as they are pharmaceutically acceptable. For example, soy bean and corn oil, in various degrees of saturation as well as beeswax, are acceptable as hydrophobic carriers for oil based formulations.

Hyrosoluble carriers include hydrosoluble polymers such as, but not limited to, polyethylene glycol (PEG), polypropylene glycol (PPG), polyethylene-propylene glycol co-polymer (PE-PPG), poly[1-(2-oxo-pyrrolidinyl)ethylene] (povidone) and polyvinyl alcohol (PVA) with different molecular weight. The terms “liquid” or “semi-solid” as used herein in the context of formulations refer to fluid materials containing drug in solution, emulsion, suspension, or paste form.

The term “pharmaceutically acceptable” means that the material being described is acceptable for use in pharmaceutical formulations by those skilled in the art. That is, any detrimental physiological effects of the material must be minimal, and even then, such detrimental effects must be substantially outweighed by the material's beneficial effects.

Trivalent metal ions used in the present invention as temporary complexing agents must be readily replaceable with divalent calcium ions after the complex passes into the intestinal track. Further, such trivalent metal ions must be pharmaceutically acceptable. Preferably, the trivalent metal ions include vanadium (V³⁺), chromium (Cr³⁺), manganese (Mn³⁺), iron (Fe³⁺) and cobalt (Co³⁺). Listed in Table 3 are typical trivalent metal ions and examples of their sources. Note, however, the scope of the present invention is not limited to metal ions in this listing.

TABLE 3
Trivalent Metal Ions

| Metal ions | Source Compounds | Metal ions | Source Compounds |
|---|---|---|---|
| Chromium | Chromic acetate | Iron | Iron sorbotex |
| | Chromic carbonate | | Ferric acetate |
| | Chromic bromide | | Ferric albuminate |
| | Chromic chloride | | Ammonium ferric |
| | | | acetate |
| | Chromic fluoride | | Ammonium ferric |
| | | | chloride |
| | Chromic formate | | Ammonium ferric |
| | | | citrate |
| | Chromic hydroxide | | Ammonium ferric |
| | | | sulfate |
| | Chromic nitrate | | Ferric bromide |
| | Ammonium chromic | | Ferric chloride |
| | sulfate | | Ferric citrate |
| | Chromium picolinate | | Ferric nitrate |
| | Chromium trioxide | | Ferric sulfate |
| | Chromium potassium | | Ferric fluoride |
| | sulfate | | Ferric formate |
| | | | Ferric oxide |
| Manganese | Manganese phosphate | Cobalt | Cobalt acetate |
| | Manganese | | Cobalt fluoride |
| | sesquioxide | | |
| | Manganese trifluoride | | Cobalt chloride |
| | Manganese trichloride | | Cobalt nitrate |
| | Manganese gluconate | | Cobaltic potassium |
| | | | nitrite |
| | Manganese sulfate | | Hexaminecobalt |
| | | | trichloride |
| Vanadium | Vanadium trifluoride | Titanium | Titanium trichloride |
| | Vanadium trisulfate | | Titanium sequisulfate |
| | Vanadium trioxide | | |
| | Vanadium trisulfide | | |
| Nickel | Nickel phosphate | | |
| | Nickel sesquioxide | | |

The term “excipient,” means any component present in a pharmaceutical formulation other than the active agent(s), complexing agent(s), or carrier(s). Examples of excipients include, but are limited to, diluents, binders, lubricants, disintegrants, fillers, pH adjusting agents, coloring agents, wetting or emulsifying agents, preservatives, and surfactants. Typically, excipients are included in a formulation to improve or enhance the characteristics of the formulation and are understood to be pharmaceutically acceptable.

Surfactants suitable for the oil based formulations of the present invention include, any surfactant known in the art for pharmaceutical or food products. For example, Tween 80 (trademark for polyoxyethylene sorbitan monooleate and Span 20 (trademark for sorbitan monolaurate) are particularly useful.

A hydrosoluble Polymer based formulation of the present invention may be conveniently prepared by mixing one or more bisphosphonates, one or more trivalent cation complexing agents, one or more hydrosoluble polymers, and optionally, one or more excipients. After mixing, the formulation is encapsulated in a soft capsule, such as a soft gelatin capsule.

In an analogous manner, a hydrophobic, i.e. oil based, formulation may be prepared by mixing one or more bisphosphonates, one or more hydrophobic carriers, one or more surfactants, and optionally, one or more excipients to form a suspension. Water can be added to the mixture if an emulsion is desired. The emulsion or suspension is then encapsulated as in an analogous manner to that described above.

A solid formulation may be prepared by any acceptable method known in the art. For example, one or more bisphosphonates, one or more trivalent cation complexing agents, and one or more excipients may be blended together to form a powder or granular mix. Alternatively, one or more bisphosphonates and one or more trivalent cation complexing agents may be mixed in water optionally at an elevated temperature. Water is removed from the resulting solution or suspension by evaporation at reduced pressure. The resulting solid complex is then optionally mixed with one or more excipients to form a powder mix or granular mix. The mix may then be compressed into a tablet or filled into a capsule according the procedure of the art. Optionally, a solid tablet core may be enrobed in a synthetic or natural polymeric coating such as a gelatin coating.

Soft gelatin capsules for oral administration of bisphosphonates are conveniently prepared by blending the active agent and excipients to form capsule filler in the form of a solution, suspension, gel, emulsion, or paste. The filler is encapsulated in a gelatin mass by methods of the art (see J. P. Stanley, The Theory and Practice of Industrial Pharmacy, Part Two, soft Gelatin Capsules). Enteric soft gelatin, as is taught in PCT application PCT/US 03/20579, may be used to encapsulate or enrobe the formulations of the present invention.

Permeability of the formulations described above was tested using the protocol taught in “Human Drug Absorption Kinetics and Comparison to Caco-2 Monolayer Permeabilities.”, E. Polli, et al., Pharm. Res., 15, 47-52 (1998). a comparative study against free bisphosphonate (BPP) product showed superior bioavailability of present bisphosphonate formulations.

The amount of the active agent in the formulations effective for treating bone loss varies with the individual and the underlying disease. However, the typical dose for treatment of bone loss is about 0.5 mg/kg to about 1.5 mg/kg of body weight administered every five to nine days. For example, a typical treatment for a 65 kg adult human would be about 70 mg of active agent administered orally once a week. Prophylactic treatment to prevent bone loss comprises administering from about 0.1 mg/kg to about 1.0 mg/kg of body weight about every two or three days. In the case of alendronate and risedronate, the dosages for prevention and treatment of bone lose are those currently approved by the FDA.

Temporary bisphosphonate-metal complexes used in the formulation taught herein were prepared and evaluated in terms of lipid solubility, cell permeability, and oral absorption in rats. Based on different formulations, bisphosphonate complexes showed 1.72 to 2.77 times the bioavailability of the parent compound when administered orally.

### EXAMPLES

The following examples are offered as illustrations of the invention and should not be construed as limitation thereto. Except where noted, conventional techniques of pharmaceutical technology are employed to prepare the formulations of the invention. Likewise, biological testing is performed in accordance with procedure described in the art except where noted.

### Example 1

### Hydrosoluble Polymer based Formulations of Alendronate in Soft Gelatin Capsules

Formulation 1 - Encapsulated suspension

| Ingredients | | mg/capsule |
|---|---|---|
| Active agent: | Alendronate | 5.0 |
| Carriers | PEG 400 | 280.0 |
| | PEG 3350 | 15.0 |
| Excipient | 0.1N aqueous HCl | 15.0 |
| Complexing agent | Hexa-ammonium cobalt chloride | 1.8 |
| Soft gelatin capsules | Gel mass | 91.0 |

Formulation 2 - Encapsulated suspension

| Ingredients | | mg/capsule |
|---|---|---|
| Active agent: | Alendronate | 10.0 |
| Carriers | PEG 600 | 435.0 |
| | PEG 3350 | 5.0 |
| Excipient | 0.1N aqueous HCl | 22.0 |
| Complexing agent | Ferric ammonium citrate | 5.3 |
| Soft gelatin capsules | Gel mass | 240.0 |

The active agent and the acidified polymeric excipient were blended to form a suspension, which was encapsulated in gelatin by methods of the art to form a soft gelatin capsule (see J. P. Stanley, The Theory and Practice of Industrial Pharmacy, Part Two, soft Gelatin Capsules.). The encapsulation was conducted using single or multiple cavity rotary dies. This method may also be used for alendronate dosages from about 1 to about 70 mg.

### Example 2

### Oil Based Emulsion Formulation of Alendronate

Encapsulated emulsion

| Ingredients | | mg/capsule |
|---|---|---|
| Active agent: | Alendronate | 5.0 |
| Excipients: | Soy bean oil | 270.0 |
| | Mixture of hydrogenated soybean oil | 36.0 |
| | and vegetable shortening | |
| | Distilled water | 18.0 |
| Surfactant | Tween 80 | 36.0 |
| Soft gelatin capsules | Gel mass | 191.0 |

The active agent was suspended in a mixture of soybean oil, hydrogenated soybean oil and vegetable oil, water, and surfactant. The resulting suspension emulsion was encapsulated in gelatin by methods of the art to form a soft gelatin capsule. (see J. P. Stanley, The Theory and Practice of Industrial Pharmacy, Part Two, soft Gelatin Capsules.)

### Example 3

### Encapsulated Oil Based Suspension Formulation of Alendronate

| Ingredients | | mg/capsule |
|---|---|---|
| Active agent: | Alendronate | 10.0 |
| Carriers | Soy bean oil | 192.0 |
| | Yellow beeswax | 24.0 |
| Excipient | Span 20 | 24.0 |
| Soft gelatin capsules | Gel mass | 91.0 |

This formulation was prepared in an analogous manner to that of Example 2. The resulting suspension was encapsulated in an enteric soft gelatin capsule for oral administration by method of the art (as in Example 2) to form a soft gelatin capsule.

### Example 4

### Tablet Formulation of Alendronate

| Ingredients | | mg/tablet |
|---|---|---|
| Active agent: | Alendronate | 5.0 |
| Excipients | Magnesium sterate | 1.0 |
| | Starch | 20.0 |
| | Sorbitol | 15.0 |
| Complexing agent | Hexa-ammonium cobalt chloride | 1.8 |

The active agent and the complexing agent are stirred in 100 ml of water. The water is removed by evaporation at reduced pressure to yield the temporary complex as a solid residue. The temporary complex and the other ingredients are blended, and then compressed into a tablet.

What is claimed is:

1. A pharmaceutical formulation comprising a therapeutic amount of a bisphosphonate or pharmaceutically acceptable salt thereof temporarily complexed with a pharmacologically acceptable complexing agent, and optionally comprising one or more pharmaceutically acceptable excipients and optionally comprising one or more pharmaceutically acceptable carriers.

2. The formulation of claim 1 wherein the formulation is a solid, solution, suspension, gel, emulsion, or paste.

3. The formulation of claim 2 wherein the formulation is a solid in the form of a powder, encapsulated powder, tablet, or enrobed tablet.

4. The formulation of claim 2 wherein the formulation is a solution, suspension, gel, emulsion, or paste contained in a soft gelatin capsule.

5. The formulation of claim 1 wherein the complexing agent comprises at least one trivalent metal ion.

6. The formulation of claim 5 wherein the trivalent metal ion is selected from trivalent vanadium, chromium, manganese, iron and cobalt.

7. The formulation of claim 4 wherein the pH of the solution, suspension, gel, or paste is about 3 to about 7.

8. The formulation of claim 1 wherein the bisphosphonate is alendronate, risedronate, etidronate, clodronate, pamidronate, tiludronate, ibandonate, zoledronate, Incadronate, olpadronate, neridronate, or amidronate.

9. The formulation of claim 6 wherein the trivalent metal ion is trivalent iron or cobalt.

10. The formulation of claim 1 which is enteric.

11. A pharmaceutical formulation comprising a therapeutic amount of a bisphosphonate or pharmaceutically salt thereof, one or more oils, one or more waxes, or a combination thereof, a surfactant, and optionally water.

12. The formulation of claim 11 wherein the bisphosphonate is alendronate, risedronate, etidronate, clodronate, pamidronate, tiludronate, ibandonate, zoledronate, Incadronate, olpadronate, neridronate, or amidronate.

13. The formulation of claim 12 wherein comprising alendronate, soy bean oil, beeswax, water and a surfactant.

14. A pharmaceutical formulation comprising a therapeutic amount of a bisphosphonate or pharmaceutically salt thereof, and optionally one or more pharmaceutically acceptable excipients and optionally one or more pharmaceutically acceptable carriers in an enteric capsule.

15. The pharmaceutical formulation of claim 14 wherein the bisphosphonate or pharmaceutically salt thereof is temporarily complexed with a pharmacologically acceptable complexing agent, and the enteric capsule is a soft gelatin capsule.

16. A method of improving the bioavailability of a bisphosphonate comprising forming a temporary complex of the bisphosphonate or pharmaceutically salt thereof with a pharmacologically acceptable complexing agent and administering the resulting complex, optionally in a formulation with one or more pharmaceutically acceptable excipients, to a patient in need thereof.

17. The method of claim 16 wherein the complexing agent comprises at least one trivalent metal ion.

18. The method of claim 17 wherein the trivalent metal ion is selected from trivalent vanadium, chromium, manganese, iron and cobalt.

19. The method of claim 16 wherein the bisphosphonate is alendronate, risedronate, etidronate, clodronate, pamidronate, tiludronate, ibandonate, zoledronate, Incadronate, olpadronate, neridronate, or amidronate.

20. The method of claim 16 wherein the bisphosphonate is alendronate, the complexing agent is trivalent iron or cobalt, and the formulation is enteric and encapsulated.

21. The method of claim 16 where formulation is encapsulated in a soft gelatin capsule.

22. An enteric soft gelatin capsule comprising an enteric gelatin shell with a filling comprised of a therapeutic amount of a bisphosphonate or pharmaceutically salt thereof temporarily complexed with a pharmacologically acceptable complexing agent, as a solution, suspension, gel, or paste, and optionally one or more pharmaceutically acceptable excipients.

23. The capsule of claim 22 wherein the complexing agent comprises at least one trivalent metal ion.

24. The capsule of claim 23 wherein the trivalent metal ion is selected from trivalent vanadium, chromium, manganese, iron and cobalt.

25. The capsule of claim 24 wherein the pH of the solution, suspension, gel, or paste is about 1 to about 7.

26. The capsule of claim 22 wherein the bisphosphonate is alendronate, risedronate, etidronate, clodronate, pamidronate, tiludronate, ibandonate, zoledronate, Incadronate, olpadronate, neridronate, or amidronate.

27. The capsule of claim 1 wherein the filling is enteric.

28. A method of treating a patient suffering from resorption of bone comprising administering to the patient a therapeutic amount of a bisphosphonate or pharmaceutically salt thereof temporarily complexed with a pharmacologically acceptable complexing agent.
